(19) United States
(12) Patent Application Publication
(10) Pub. No.: US 2017/0209335 A1
(43) Pub. Date: Jul. 27, 2017

(54) Physiological State Feedback System and Control Method Thereof

(71) Applicants: Chao-Yu Yueh, Taipei City (TW); Oivita Creative Co., Ltd., Taipei City (TW)

(72) Inventor: Chao-Yu Yueh, Taipei City (TW)

(21) Appl. No.: 15/409,640

(22) Filed: Jan. 19, 2017

(30) Foreign Application Priority Data
Jan. 22, 2016 (TW) 105102002

(51) Int. Cl. A61H 19/00; A61B 5/00; A61B 5/145; A61B 5/021; A61B 5/024

(52) U.S. Cl. CPC A61H 19/50; A61B 5/021; A61B 5/02438; A61B 5/14542; A61H 19/32; A61B 5/4368

(57) ABSTRACT

A physiological state feedback system includes a physiological state detecting device, an electric massage device and a portable processing device. The physiological state detecting device is worn on a user, and detects to output a physiological information signal according to a state of the user. The electric massage device outputs a device motion signal. The portable processing device receives the physiological information signal and the device motion signal via wireless transmission technology to produce a feedback signal.

### CROSS REFERENCE TO RELATED APPLICATIONS

This Non-provisional application claims priority under 35 U.S.C. §119(a) on Patent Application No. 105102002 filed in Taiwan, Republic of China on Jan. 22, 2016, the entire contents of which are hereby incorporated by reference.

### BACKGROUND

1. Technical Field

The disclosure relates to a feedback system, in particular, to a physiological state feedback system and control method thereof.

2. Description of Related Art

In sexual life, the woman often accompanied by muscle contraction, aerobic metabolism to speed up, resulting in respiratory and heart rate, and blood pressure phenomenon when during the state of orgasm, due to the central nervous system of the human body is in the excitatory state. When orgasm, the respiratory rate may be up to 40 times per minute, heart rate increased to 120 times per minute, or even up to 150 times per minute to 160 times. To achieve orgasm, besides the physiological stimuli, the psychological level of stimulation is also very important.

With the rapid development of electronic technology, various products have also been developed in the field of sexual assistive devices, such as electric massagers and vibration rings. However, these products are individually acted alone in the improper use of the state may cause discomfort to each other.

It is therefore an important subject to provide a physiological state feedback system and its control method capable of timely observing the physiological state of the partner in the sexual behavior so as to further increase the enjoyment of life and to be appropriately relaxed.

### SUMMARY OF THE INVENTION

In view of the foregoing, the invention is to provide a physiological state feedback system and its control method, which can provide instant feedback information during a sexual behavior so that users can observe the physiological state with one another to increase interest of sexual life.

To achieve the above, the present application is to provide a physiological state feedback system, which includes a physiological state detecting device, an electric massage device and a portable processing device. The physiological state detecting device is to detect a state of a human body and to output a physiological information signal in accordance with the detection result. The electric massage device is to output an action information signal in accordance with an action mode of the electric massage device. The portable processing device is to generate a feedback signal in accordance with the physiological information signal and the action information signal.

To achieve the above, the present application is to provide a control method of a physiological state feedback system, which is cooperating with an electric massage device, a physiological state detecting device and a portable processing device. The control method includes the following steps. Step S01 is pairing the electric massage device, the physiological state detecting device and the portable processing device to each other. Step S02 is enabling the electric massage device to vibrate with a frequency and an amplitude and to output an active information signal. Step S03 is enabling the physiological state detecting device to output a physiological information signal according to a detecting result. Step S04 is receiving the active information signal and the physiological information signal by the portable processing device. Step S05 is generating a feedback signal by comparing the active information signal, the physiological information signal and a predetermined value by the portable processing device.

As mentioned above, the physiological state feedback system and its control method utilizes the portable processing device to receive the physiological information signal and the active information signal, respectively, and to generate the feedback signal to inform the users. In addition, the portable processing device also can control the active mode of the electric massage device so as to increase the interest of sexual life.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The parts in the drawings are not necessarily drawn to scale, the emphasis instead being placed upon clearly illustrating the principles of at least one embodiment. In the drawings, like reference numerals designate corresponding parts throughout the various diagrams, and all the diagrams are schematic.

FIG. 1 is schematic block showing a physiological state feedback system according to the embodiment of the invention.

FIG. 2 is a flow chart showing a control method of a physiological state feedback system according to the embodiment of the invention.

### DETAILED DESCRIPTION

The parts in the drawings are not necessarily drawn to scale, the emphasis instead being placed upon clearly illustrating the principles of at least one embodiment. In the drawings, like reference numerals designate corresponding parts throughout the various diagrams, and all the diagrams are schematic.

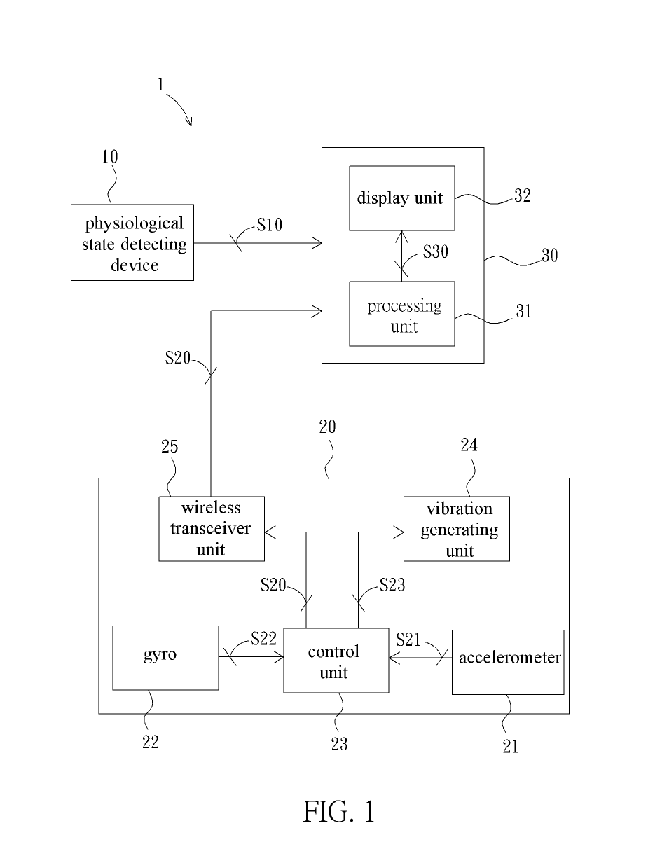

Referring to FIG. 1, a physiological state feedback system 1 includes a physiological state detecting device 10, an electric massage device 20 and a portable processing device 30. In the embodiment, the physiological state detecting device 10 is to wear with a woman, and the electric massage device 20 is to wear with a man. The portable processing device 30 can be paired with the physiological state detecting device 10 and the electric massage device 20 so as to build a closed network to transmit the signal to each other. In the embodiment, the portable processing device 30 can be paired with the physiological state detecting device 10 and the electric massage device 20 by the wireless technology, for example the Bluetooth technology or the Near Field Communication (NFC) technology.

The physiological state detecting device 10 may be a wellness product (e.g. activity tracker with heart rate, blood pressure or blood oxygen). In the embodiment, the wellness product is to generate a physiological information signal S10 by an optical measurement technology. To gauge the heart rate of the user, the physiological state detecting device 10 flashes green light at the skin and records the amount of this light the blood absorbs.

The electric massage device 20 has an accelerometer 21, a gyro 22, a control unit 23, a vibration generating unit 24 and a wireless transceiver unit 25. The electric massage device 20 is to output an active information signal S20. The active information signal S20 includes a force signal S21 and a direction signal S22.

The force signal S21 is generated from the accelerometer 21 while the electric massage device 20 is movement. The level of the force signal S21 is defined by the acceleration of the movement of the electric massage device 20 due to the different acceleration causes a different intensity. In the embodiment, the accelerometer 21 may be a three-axis accelerometer that can track the forces along the three axial.

The direction signal S22 is generated from the gyro 22 while the electric massage device 20 is movement. In the embodiment, the gyro 22 may be a three-axis gyro that can measure the angular velocity along the three axial.

The control unit 23 is electrically connected to the accelerometer 21 and the gyro 22. The control unit 23 is to receive the force signal S21 and the direction signal S22 so as to integrate as the active information signal and to output a control signal S23. In the embodiment, the control unit 23 can be a microprocessor or a Central Processing Unit (CPU).

The vibration generating unit 24 is electrically connected to the control unit 23. The vibration generating unit 24 is to generate an vibration wave according to the control signal S23 to vibrate the electric massage device 20. In the embodiment, the vibration generating unit 24 may be a vibration motor, wherein the frequency and amplitude of the vibration wave can be modulated by the control signal S23.

The wireless transceiver 25 is electrically connected to the control unit 23 for receiving and delivering the active information signal S20. In the embodiment, the active information signal S20 may be delivered to the portable processing device 30 by the blue tooth technology, the NFC technology, the WiFi technology or the Zigbee technology.

The portable processing device 30 is to receive the physiological information signal S10 and the active information signal S20 by the wireless technology. The portable processing device 30 has a processing unit 31 for generating a feedback signal S30 in accordance with the physiological information signal S10 and the active information signal S20. The feedback signal S30 can be displayed on a display unit 32 of the portable processing device 30. In other embodiment, the feedback signal S30 can be shown by the type of sound, voice or lights. In addition, the control unit 23 of the electric massage device 20 can generate the control signal S23 in accordance with the feedback signal S30 so as to modulate the frequency and/or the amplitude of the vibration wave generated by the vibration generating unit 24.

In the embodiment, the physiological state detecting device 10 may be a heart rate detecting device, the electric massage device 20 may be a vibrating ring, and the portable processing device 30 may be a smart phone. In a first time to use the physiological state feedback system, the smart phone have to pair to the heart rate detecting device and the vibrating ring.

The heart rate detecting device can be worn on a wrist of a woman, and the vibrating ring can be worn around a genital of a man. In the embodiment, when used by a couple, the smart vibrating ring will be placed around the penis and be positioned in the right place so that both you and your partner get the full sensation of vibration. The heart rate detecting device is to transmit the heart rate signal to the smart phone, and the vibrating ring is to transmit the force signal and the direction signal to the smart phone during the sexual behavior.

The application program in the smart phone can integrate the heart rate signal, force signal and the direction signal so as to generate a feedback signal to display on a display of the smart phone. In the embodiment, the application program is programed with a smart algorithm to analyze all the information in real time. In the embodiment, the feedback signal represents a level of the female orgasm that can be obtained by a look-up table. In addition, the feedback signal can be transmitted to the vibrating ring so as to change the frequency or the amplitude of the vibrating ring.

Furthermore, the relationship between the physiological state of the female user and her orgasm can be recorded as a relative record by a learning mode of the application program. The relative record includes the force strength, the motion direction, the vibrating frequency, the vibrating amplitude of the vibrating ring and the physiological state of female user.

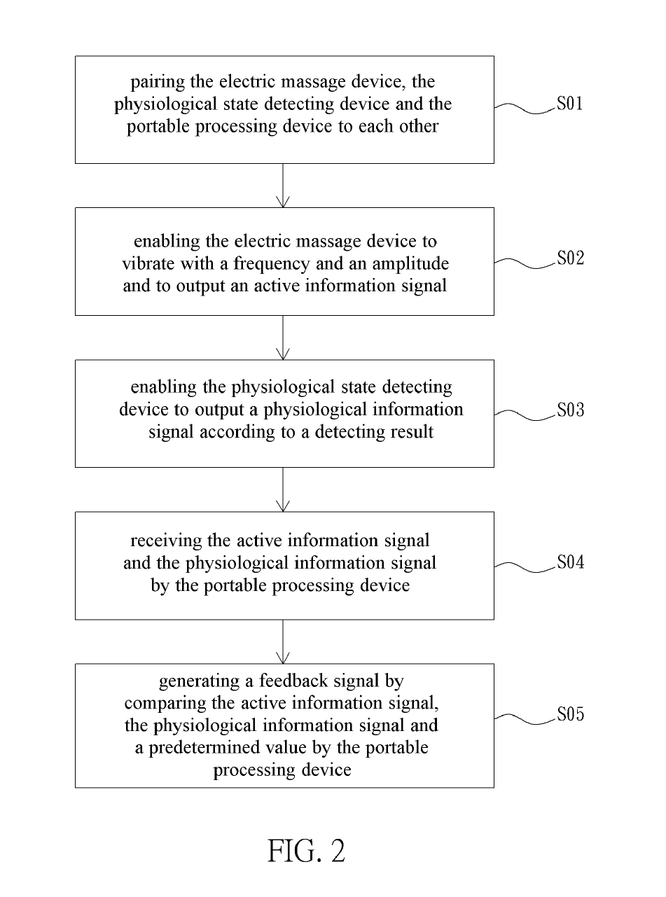

Referring to FIG. 2, a control method of a physiological state feedback system is cooperating with an electric massage device, a physiological state detecting device and a portable processing device. The control method includes the following steps.

Step S01 is pairing the electric massage device, the physiological state detecting device and the portable processing device to each other. The electric massage device, the physiological state detecting device and the portable processing device can be paired by the Bluetooth technology or the NFC technology.

Step S02 is enabling the electric massage device to vibrate with frequency and amplitude and to output an active information signal.

Step S03 is enabling the physiological state detecting device to output a physiological information signal according to a detecting result.

Step S04 is receiving the active information signal and the physiological information signal by the portable processing device.

Step S05 is generating a feedback signal by comparing the active information signal, the physiological information signal and a predetermined value by the portable processing device. In the embodiment, the predetermined value is a variation amount of the physiological information signal corresponding to the timeline.

As described above, the predetermined value may be modified under the learning mode of the application program. In the embodiment, the predetermined value may be a specific slope of variation amount of the heart rate. The predetermined value can be recorded by the user or automatic recorded by the application program.

As mentioned above, the physiological state feedback system and its control method is to enhance female orgasm experience and thus bring joy to both sexes. The physiological state feedback system utilizes the portable processing device to receive and integrate the physiological information signal and the active information signal so as to generate the feedback signal to inform the users to increase interest of sexual life.

Even though numerous characteristics and advantages of certain inventive embodiments have been set out in the foregoing description, together with details of the structures and functions of the embodiments, the disclosure is illustrative only. Changes may be made in detail, especially in matters of arrangement of parts, within the principles of the present disclosure to the full extent indicated by the broad general meaning of the terms in which the appended claims are expressed.

What is claimed is:

1. A physiological state feedback system, comprising:
a physiological state detecting device that is detecting a state of a user and outputting a physiological information signal according to the detection result;
an electric massage device that is outputting an action information signal according to an action mode; and
a portable processing device that is generating a feedback signal according to the physiological information signal and the action information signal.

2. The physiological state feedback system defined in claim 1, wherein the physiological information signal includes at least one of a heart rate information, a blood pressure information and a blood oxygen concentration information.

3. The physiological state feedback system defined in claim 1, wherein the action information signal includes at least one of a force signal and a direction signal.

4. The physiological state feedback system defined in claim 3, wherein the electric massage device comprising:
an accelerometer that is outputting the force signal;
a gyro that is outputting the direction signal;
a control unit that is electrically connected to the accelerometer and the gyro for receiving the force signal and the direction signal so as to output a control signal;
an vibration generating unit that is electrically connected to the processor for receiving the control signal so as to generate an vibration wave; and
a wireless transceiver unit that is electrically connected to the control unit for receiving and delivering the force signal and the direction signal.

5. The physiological state feedback system defined in claim 4, wherein the wireless transceiver unit further receives the feedback signal delivered by the portable processing device.

6. The physiological state feedback system defined in claim 5, wherein the control unit controls the frequency and/or amplitude of the vibration wave generated from the vibration generating unit according to the feedback signal.

7. A control method of a physiological state feedback system that is cooperating with an electric massage device, a physiological state detecting device and a portable processing device, comprising:
pairing the electric massage device, the physiological state detecting device and the portable processing device to each other;
enabling the electric massage device to vibrate with a frequency and an amplitude and to output an active information signal;
enabling the physiological state detecting device to output a physiological information signal according to a detecting result;
receiving the active information signal and the physiological information signal by the portable processing device; and
generating a feedback signal by comparing the active information signal, the physiological information signal and a predetermined value by the portable processing device.

8. The physiological state feedback system defined in claim 7, wherein the predetermined value is a variation amount of the physiological information signal corresponding to the timeline.
